(19) United States
(12) Patent Application Publication
(10) Pub. No.: US 2005/0153298 A1
(43) Pub. Date: Jul. 14, 2005

(54) Protein micro-arrays and multi-layered affinity interaction detection

(76) Inventors: Dmitry S. Gembitsky, Bloomfield, NJ (US); Paul Tempst, New York, NY (US)

Correspondence Address: Benjamin Adler;Adler & Associates, 8011 Candle Lane, Houston, TX, 77071, US

(21) Appl. No.: 10/521,712

(22) PCT Filed: Oct. 23, 2002
(86) PCT No.: PCT/US02/33917

Related U.S. Application Data
(60) Provisional application No. 60/335,645, filed on Oct. 23, 2001.

(51) Int. Cl. C12Q 1/68; G01N 33/53; G06F 19/00; G01N 33/48; G01N 33/50

(52) U.S. Cl. 435/6; 435/7.1; 702/19

(57) ABSTRACT

The present invention provides proteomic techniques that extend sensitive and quantitative analysis of proteins to post-translational modifications. Protein micro-arrays and/or multiplex coded-microbeads are used in combination with multilayered affinity interaction detection (MAID) methods that permit high throughput analysis of cellular protein modifications and functional protein interactions.

### CROSS-REFERENCE TO RELATED APPLICATION

This application claims benefit of priority of provisional application U.S. Ser. No. 60/335,645, filed Oct. 23, 2001, now abandoned.

### BACKGROUND OF THE INVENTION

1. Field of the Invention

The present invention relates generally to the field of proteomics. More specifically, the present invention relates to protein micro-arrays and multi-layered affinity interaction detection procedures that allow high throughput and quantitative cellular protein profiling.

2. Description of the Related Art

Completion of the human genome project has brought with it a new round of challenges to characterize the components and understand the behavior of a cell. Protein science will play a major role in this endeavor because proteins carry out most of the work in a cell, including control of growth and development. While genomics and functional genomics will continue to provide significant insights, it is likely to overlook many other critical aspects because levels of protein expression, type and extent of post-translational modifications, as well as multi-protein-protein interactions are not probed directly. Advanced proteomic techniques, for the most part still to be developed, will therefore become central research tools over the next decade in complementing existing ones for genomic analysis.

The goal of proteomics is to perform global analysis of changes in both the quantity and post-translational modifications of all proteins in a cell, as well as to analyze the network of protein-protein interactions. Changes in the proteome may be brought about either by growth, differentiation, senescence, exposure to bioactive agents, or genetic alteration. The most common approach for global analysis of protein expression to date is by 2D-gel electrophoretic display and high-throughput mass spectrometric (MS) identification. This is intended to be, or to become, the protein equivalent of expression profiling by DNA micro-arrays. However, compared to micro-arrays, the throughput and sensitivity of 2D-PAGE/MS analysis are orders of magnitude lower, and many technical and practical problems remain unsolved. Moreover, post-translational modification analysis is either very poorly, or not at all, addressed by the 2D-PAGE/MS-ID approach.

Identifying dynamic covalent protein modifications in their proper biological context is clearly a biochemical problem. for example, reversible phosphorylation, one of dozens or hundreds of different estimated modifications, is critical for transmission of signals in all living cells. Not surprisingly, deregulation of reversible phosphorylation has been implicated in disease including cancer. This raises the question of which proteins are modified, and how, where and when they are modified. Analysis can only be done at the protein level, and it will involve high-throughput identifications at the highest levels of sensitivity. Similar issues and questions can be raised for analysis of protein interactions.

Several labs are currently trying to put specific antibodies, each against a different protein, onto micro-arrays (1, WO 00/63701). The idea is to carry out expression profiling, DNA array-style, at the protein level. This particular approach may eventually be better suited for global protein analysis than the intricate 2D-PAGE/MS scheme, for reasons of better throughput (massive parallel detection), sensitivity (microspot laser fluorescence) and dynamic range. Most of the technologic difficulties in creating these chips have already been solved. However, it will take great effort and expense to produce 35,000 unique antibodies that are absolutely specific in recognition of their cognate human protein targets.

Predictably, this effort will start with designer chips, containing several hundred to a few thousand selected antibodies. By establishing international, large-scale antibody (including phage display antibodies) producing consortia, this challenge can almost certainly be met. In fact, the effort is quite comparable to sequencing 3 billion base pairs in the human genome project. The difference being that, once complete protein chips are available, the proteome project will only begin, not end, as there are infinite number of proteomic snapshots to be taken from many different cell types.

Two potential problems are evident. First, antibody-antigen interactions are likely to be quite variable, making general ‘capturing’ conditions for 35,000 different proteins difficult to establish. Second, entire protein complexes may bind to a single antibody by virtue of interaction with the targeted antigen. Because a number of cellular proteins will be fluorescently labeled, this could lead to over-representation of the amount of any particular antigen, creating a false positive-like situation. Complexes must therefore be disrupted under conditions that don't interfere with binding to the immobilized antibodies. Both challenges can conceivably be met over time. Thus, protein chips will present great enabling technologies for cell analysis. Both concept and execution are straightforward, albeit very laborious and time consuming to get the necessary tools (2).

Alternatively, optically encoded microbeads (<b 3 micron) could be used instead of micro-arrays for highly parallel, quantitative analysis of proteins (and other molecules). Multicolor coded beads are uniquely identifiable, for instance in a miniaturized fluorescence-activated cell sorter, through a combination of wavelength and intensity multiplexing embedded inside each bead. Analogous to the spatially resolved, x,y coordinate-coded spots on planar chips, each microbead would contain a single monoclonal antibody on its surface against a specific human protein. In this scenario, at least 35,000 beads would be needed. Cellular proteins, which are fluorescently labeled with no spectral overlap with the ‘tags’, are bound to the beads. Each protein will bind to a specific bead bearing the corresponding antibody, thus providing the quantitative aspect for profiling (3).

U.S. Pat. No. 6,329,209 and U.S. Pat. No. 6,365,418 disclose arrays of biomolecules or multimolecular complexes, i.e., protein capture agents, which bind a molecule to itself and methods of making such arrays. These protein capture agents can specifically bind an expression product or fragment thereof from a cell or a population of cells. The amount or presence of the expression product bound to a capture agent can be detected directly or indirectly.

However, even though the above techniques are perfectly optimized, neither approach will provide any direct information on dynamic protein modifications, or protein-protein, protein-nucleic acid and protein-small molecule interactions in the cell. The inventors have recognized an increased need for efficient throughput for protein profiling as a tool in cell analysis. The prior art is deficient in proteomic techniques that allow sensitive and high throughput analysis of protein modifications and interactions. Specifically, the art is deficient in the lack of expression profiling that allows parallel quantitation of all proteins expressed in a cell or tissue; modification proteomics that analyzes the type, degree and timing of dynamic post-translational protein modifications; and interaction proteomics that examines functional protein interactions. The present invention fulfills this long-standing need and desire in the art.

### SUMMARY OF THE INVENTION

In one embodiment of the present invention there is provided a high throughput and quantitative method of analyzing post-translational protein modifications in a sample comprising the steps of preparing at least one of N identical arrays of immobilized protein capture agents, each of the capture agents binding specifically to a protein in the sample; and performing in any order the steps of applying the proteins of the sample to at least one of the N arrays of immobilized protein capture agents; and binding the proteins of the sample to at least one of X detectable affinity reagents to label the proteins, where X is an integer from 1 to N and where each of the X detectable affinity reagents specifically recognizes one of N post-translational protein modifications and measuring a signal associated with the detectable affinity reagents, wherein quantitation of the signal of the X detectable affinity reagent(s) provides a high throughput and quantitative analysis of post-translational protein modifications in the sample.

In another embodiment of the present invention there is provided a high throughput and quantitative method of comparative analysis of post-translational protein modifications in different samples, comprising the steps of preparing an array of immobilized protein capture agents, where each of the capture agents binds specifically to a protein in the samples; incubating a first sample A with an affinity reagent, where the affinity reagent is labeled with a first detectable label and where the affinity reagent specifically recognizes a post-translational protein modification; incubating a second sample B with the affinity reagent, where the affinity reagent is labeled with a second detectable label and applying a mixture of the affinity reagent-labeled samples A and B to the array of immobilized protein capture agents. The relative signals from the first and the second detectable labels on the affinity reagents are quantified such that ratios of these relative signals of the first and the second detectable labels correlate to the relative abundance of the post-translational modifications between sample A and sample B.

In another embodiment of the present invention there is provided a high throughput and quantitative method of comparative analysis of post-translational protein modifications in different samples, comprising the steps of preparing an array of immobilized protein capture agents, where each of the capture agents binds specifically to a protein in the samples; incubating a first sample A with an affinity reagent labeled with a first fluorophore, where the affinity reagent specifically recognizes a post-translational protein modification; incubating a second sample B with the affinity reagent labeled with a second fluorophore; applying a mixture of the affinity reagent-labeled samples A and B to the array of immobilized protein capture agents; measuring the fluorescence emission of the first and the second fluorophores, and calculating the ratios of relative fluorescence of the first and the second fluorophores, where the ratios correlate to the relative abundance of the post-translational modifications between sample A and sample B.

In yet another embodiment of the present invention there is provided a high throughput and quantitative method of analyzing protein interactions, comprising the steps of preparing an array of immobilized protein capture agents, where each of the capture agents binds specifically to a protein in the sample; labeling the proteins in the sample with a first fluorophore; applying the labeled proteins to the array of immobilized protein capture agents; labeling molecules with a second fluorophore; applying the labeled molecules to the labeled proteins captured on the array of immobilized capture agents, where the molecules specifically bind to the labeled proteins captured on the array of immobilized capture agents; and measuring the emission of the first and second fluorophores, where the relative fluorescence of the first and of the second fluorophores correlates with an interaction of the molecules with the proteins thereby providing high throughput and quantitative analysis of the protein interactions.

In still another embodiment of the present invention there is provided a kit for a high throughput and quantitative method of analyzing post-translational protein modifications comprising at least one array of immobilized protein capture agents; at least one buffer medium; and at least one affinity reagent where each of the affinity reagents recognizes a specific post-translational protein modification.

In still yet another embodiment of the present invention there are provided kits for a high throughput and quantitative method of analyzing post-translational protein modifications comprising a set of buffer media or at least one affinity reagent and at least one buffer medium or at least one array of immobilized protein capture agents and at least one buffer medium.

Other and further aspects, features, and advantages of the present invention will be apparent from the following description of the presently preferred embodiments of the invention. These embodiments are given for the purpose of disclosure.

### BRIEF DESCRIPTION OF DRAWINGS

So that the matter in which the above-recited features, advantages and objects of the invention, as well as others that will become clear, are attained and can be understood in detail, more particular descriptions of the invention briefly summarized above may be held by reference to certain embodiments thereof that are illustrated in the appended drawings. These drawings form a part of the specification. It is to be noted, however, that the appended drawings illustrate preferred embodiments of the invention and therefore are not to be considered limiting in their scope.

FIG. 1A demonstrates quality control of micro-array printing procedure, deposited Cy⁵-labeled IgG is shown by arrows.

FIG. 1B demonstrates verification of retention of antibodies on the array, deposited non-labeled mouse IgG was visualized by Cy⁵-labeled goat anti-mouse antibody at the completion of an experiment (shown by arrow).

FIG. 1C depicts an array of 21 capture antibodies after incubation with Cy⁵-labeled protein extract, spots corresponding to anti-Raf-1 antibody are shown by arrow.

FIG. 1D depicts an array of 21 capture antibodies after detection of phospho-Tyr proteins, spots corresponding to anti-Raf-1 antibody are shown by arrow.

FIG. 2A depicts the quantification of protein expression from first virtual layer.

FIG. 2B depicts the quantification of protein phosphorylation at Tyr residues from the second virtual layer.

FIG. 2C summarizes the relative expression and phosphorylation at Tyr residues of proteins as a virtual overlay.

### DETAILED DESCRIPTION OF THE INVENTION

In one embodiment of the present invention there is provided a high throughput and quantitative method of analyzing post-translational protein modifications in a sample comprising the steps of preparing at least one of N identical arrays of immobilized protein capture agents, each of the capture agents binding specifically to a protein in the sample; and performing in any order the steps of applying the proteins of the sample to at least one of the N arrays of immobilized protein capture agents; and binding the proteins of the sample to at least one of X detectable affinity reagents to label the proteins, wherein X is an integer from 1 to N and where each of the X detectable affinity reagents specifically recognizes one of N post-translational protein modifications and measuring a signal associated with the detectable affinity reagents, where quantitation of the signal from said X detectable affinity reagent(s) provides a high throughput and quantitative analysis of post-translational protein modifications in the sample.

In one aspect of this embodiment the proteins of the sample are bound to X detectable affinity reagents to label the proteins, the labeled proteins are applied to at least one of the N arrays of immobilized protein capture agents; and the labeled proteins captured in at least one of the N arrays are bound to (N-X) of the detectable affinity reagents. In another aspect, the proteins of the sample are bound to all of the X detectable affinity reagents to label them and then applied to at least one of the N arrays of immobilized protein capture agents. In a third aspect of this embodiment the proteins of the sample are applied to at least one of the N arrays of immobilized protein capture agents and then bound to all of the X detectable affinity reagents.

In all aspects of this embodiment the protein capture agents and the affinity reagents may be an antibody, an antibody fragment, a recombinant protein, a nucleic acid or a phage particle. Each of the X affinity reagents may be detectably distinct from the first affinity reagent and from each other. Alternatively, if one of each of a second through N affinity reagents is applied separately to the labeled proteins captured in one each of the identical N arrays then the affinity reagents may be detectably identical.

Further to these aspects the affinity reagents may be labeled with a detectable tag. Representative examples are a fluorophore, biotin, streptavidin, an enzyme, a radioactive isotope, or oligonucleotide. Alternatively, the affinity reagents may be labeled with secondary detectable affinity reagents. The secondary affinity reagents may also be an antibody, an antibody fragment, a recombinant protein, a nucleic acid or phage particle.

In another embodiment of the present invention there is provided a high throughput and quantitative method of comparative analysis of post-translational protein modifications in different samples, comprising the steps of preparing an array of immobilized protein capture agents, where each of the capture agents binds specifically to a protein in the samples; incubating a first sample A with an affinity reagent, where the affinity reagent is labeled with a first detectable label and where the affinity reagent specifically recognizes a post-translational protein modification; incubating a second sample B with the affinity reagent, where the affinity reagent is labeled with a second detectable label and applying a mixture of the affinity reagent-labeled samples A and B to the array of immobilized protein capture agents. The relative signals from the first and the second detectable labels on the affinity reagents are quantified such that ratios of these relative signals from the first and the second detectable labels correlate to the relative abundance of the post-translational modifications between sample A and sample B. The detectable labels may be fluorophores, nucleic acids or enzymes. In this embodiment the protein capture agents and the affinity reagents are as described supra.

In yet another embodiment of the present invention there is provided a high throughput and quantitative method of comparative analysis of post-translational protein modifications in different samples, comprising the steps of preparing an array of immobilized protein capture agents, where each of the capture agents binds specifically to a protein in the samples; incubating a first sample A with an affinity reagent labeled with a first fluorophore, where the affinity reagent specifically recognizes a post-translational protein modification; incubating a second sample B with the affinity reagent labeled with a second fluorophore; applying a mixture of the affinity reagent-labeled samples A and B to the array of immobilized protein capture agents; measuring the fluorescence emission of the first and the second fluorophores, and calculating the ratios of relative fluorescence of the first and the second fluorophores, where the ratios correlate to the relative abundance of the post-translational modifications between sample A and sample B. In this embodiment the protein capture agents and the affinity reagents are as described supra.

In yet another embodiment of the present invention there is provided a high throughput and quantitative method of analyzing protein interactions, comprising the steps of preparing an array of immobilized protein capture agents, where each of the capture agents binds specifically to a protein in the sample; labeling the proteins in the sample with a first fluorophore; applying the labeled proteins to the array of immobilized protein capture agents; labeling molecules with a second fluorophore; applying the labeled molecules to the labeled proteins captured on the array of immobilized capture agents, where the molecules specifically bind to the labeled proteins captured on the array of immobilized capture agents; and measuring the emission of the first and second fluorophores, where the relative fluorescence of the first and of the second fluorophores correlates with an interaction of the molecules with the proteins thereby providing high throughput and quantitative analysis of the protein interactions. In this embodiment the protein capture agents and the affinity reagents are as described supra.

In still another embodiment of the present invention there is provided a kit for a high throughput and quantitative method of analyzing post-translational protein modifications comprising at least one array of immobilized protein capture agents; at least one buffer medium; and at least one affinity reagent where each of the affinity reagents recognizes a specific post-translational protein modification. In this embodiment the protein capture agents and the affinity reagents and the types of labels on the affinity reagents are as described supra.

In still yet another embodiment of the present invention there are provided kits for a high throughput and quantitative method of analyzing post-translational protein modifications comprising a set of buffer media or at least one affinity reagent and at least one buffer medium or at least one array of immobilized protein capture agents and at least one buffer medium. Again in this embodiment the protein capture agents and the affinity reagents and the types of labels on the affinity reagents are as described supra.

The present invention is directed to protein micro-arrays and multi-layered affinity interaction detection (MAID) procedures that will allow all of the above aspects of cellular protein profiling to be performed in hours rather than months or years it would take with the current technology of gel-display/mass spectrometric identification. The multi-layered affinity interaction detection procedures offer better prospects for automation, throughput, sensitivity, quantitation and dynamic range. It will likely become an important tool in the proteomics drug research market as genechips are in the genomics industry today. The process and reagents described herein are for the purpose of whole-cell or tissue profiling of any or all modifications of all proteins in such cell or tissue and for profiling of protein-protein, protein-DNA and protein-small molecule interactions when chips of protein capture agents and/or coded affinity beads are brought to the level and complexity of adequately profiling 35,000 or more different proteins in a cell.

In contemplating modification proteomics, post-translational protein modifications can be specifically detected by suitable affinity reagents such as monoclonal antibodies or affinity-recognizable tags, e.g. biotin—recognized by streptavidin incorporated by specific chemical reactions. These approaches have already been shown to be workable strategies, as examples of both high-specificity antibodies against phospho-tyrosine (3) and high-specificity chemical tagging of nitroso-cysteine (4) are available in the literature. Further examples of post-translational modifications include, but are not limited to, phopsho-serine, phospho-threonine, phospho-histidine, N-acetyl-lysine, N-acetyl-arginine, N-methyl-lysine, N-methyl-arginine, N-acetyl glucosamine (GlcNac)-serine, GlcNac-threonine, sulfo-tyrosine and nitroso-tyrosine.

One object of the present invention is aimed at developing (i) monoclonal antibodies against various other post-translational modifications, and (ii) chemical tagging techniques for various other or the same modifications. The critical factors in developing these reagents and methods are: (i) a particular type of modification should be recognized or tagged in all cellular proteins whenever it is present; (ii) no other chemical moieties, whether part of the proteins or not, should be recognized or tagged; and (iii) recognition/tagging should be independent of the surrounding amino acid sequence, i.e., the epitope or reactivity should be exclusively limited to the modifying group.

The new reagents/tagging methods disclosed herein serve to identify modified cellular proteins that have already been captured in a first round of profiling and are held in place through either affinity forces or post-capture covalent linkage on discrete spots of a protein chip or on a particular coded microbead. For example, a microchip with 10,000 monoclonal antibodies against 10,000 different proteins is used for whole-cell protein profiling of a particular type of blood cell and captures 8543 labeled proteins providing a quantitative read-out.

Continuing with this example, in a second round or ‘layer’ of affinity interaction, a differently labeled monoclonal antibody (mAb) against phospho-tyrosine (P-Tyr) is used to screen this chip again and 1094 of those 8543 proteins are detected as having P-Tyr modification. Since these proteins are attached to x,y-spatially encoded targets, their identities can be determined immediately, thus making large scale parallel identification possible. Furthermore, and very importantly, if the anti-P-Tyr monoclonal antibody is fluorescently labeled with no spectral overlap with the cellular protein label, a quantitative read-out is possible of the modification of any protein on a per mole basis. Fluorophore ‘A’ provides quantitation of the amount of protein ‘x’ bound per spot, whereas fluorophore ‘B’ provides quantitation of how much modification ‘y’ is present per spot, and the ratio of B/A will provide a measure of how extensively protein ‘x’ is modified with ‘y’.

The above scenario lends itself to further multiplexing using different monoclonal antibodies with specificities for different post-translational modifications. These monoclonal antibodies are all labeled with spectrally distinguishable fluorophores, which may be a combination of excitation and emission spectra, that are monitored at different wavelengths or by using a diode-array detector. In cases where a chemical affinity-tagging procedure was used, the corresponding interacting protein, e.g. streptavidin for a biotin-tag, would also contain a color-coded label for facile detection/quantitation. Consequently, profiling of tens of thousands of cellular proteins can be done simultaneously for dozens of different modifications in a single experiment, providing hundreds of thousands of data points in an analytical feat that cannot conceivably be achieved by any type of mass spectrometric analysis.

The multi-layered affinity interaction detection (MAID) procedure can be used for comparative analysis of protein pools from two different cell populations; e.g. cancer cells versus normal ones or growth factor stimulated versus unstimulated cells, etc. In analogy with cDNA microarray procedures, a two-color, e.g. green and red, system may be used to detect differentially modified proteins in each pool. For example any particular monoclonal antibody, such as anti-P-Tyr monoclonal antibody, is labeled with an appropriate dye ‘Green’, and in a separate batch with dye. ‘Red’. Batch ‘G’ is mixed with the protein pool from cell I and batch ‘R’ is incubated with proteins from cell II. Any protein containing a P-Tyr will have labeled monoclonal antibody bound to it, i.e. monoclonal antibody-G to I and monoclonal antibody-R to II. The reaction should proceed to completion.

Both pools are then combined and placed on a chip of immobilized protein capture agents as described above. After each of the proteins, some of which have nonoclonal antibody-G or monoclonal antibody-R bound to them while most others do not, have bound to their cognate antibodies on the array, the relative abundances of the P-Tyr, or other, modification between the pools are quantified by calculating the ratio of the two fluorescent signals. If the ratio is to be corrected for protein abundance, i.e., modification on per mole basis, two more spectrally non-overlapping dyes will be needed to quantitate relative abundance for all proteins.

In a preferred embodiment the present invention is directed to a high throughput and quantitative method of analyzing post-translational protein modifications in a sample. First, fluorophore-labeled proteins are applied to an array of immobilized first antibodies that binds specifically to the proteins. Then, second antibodies that are labeled with a second fluorophore are applied to the proteins captured on the array of first antibodies. These second antibodies specifically recognize a post-translational protein modification present on the captured proteins. Measuring the emission of the first and second fluorophores would provide high throughput and quantitative analysis of post-translational protein modifications in the sample. The above method may further comprise the step of applying third antibodies that are labeled with a third fluorophore to the captured proteins, wherein the third antibodies specifically recognize a second post-translational protein modification.

The above method may be executed on separate identical micro-arrays of protein capture agents with subsequent computer analysis, e.g., virtual overlay. Virtual multi-layered affinity interaction detection will allow usage of the same fluorophore in all virtual layers and will be compatible with most available scanning hardware. Alternatively, the second, third, fourth and so on antibodies can be free of a fluorescent tag. They can be detected by numerous alternate techniques that are well known to one having ordinary skill in the art. Those include, but not limited to labeling with biotin, horseradish peroxidase, alkaline phosphatase, oligonucleotides, streptavidin and application of secondary antibodies or antibody fragments that can be detected in a similar fashion or directly conjugated to a fluorophore.

The present invention is also directed to a high throughput and quantitative method of analyzing protein interactions. For example, in a first step, fluorophore-labeled proteins are applied to an array of immobilized antibodies that binds specifically to the proteins. Then molecules that are labeled with a second fluorophore are applied to the proteins captured on the array of immobilized antibodies. These molecules would bind specifically to the captured proteins. Measuring the emission of the first and second fluorophores would provide high throughput and quantitative analysis of protein interactions. Representative examples of molecules useful in this assay include protein molecules, small molecules, drug molecules or nucleic acid molecules.

Thus, any types of protein interactions can be similarly analyzed by methods disclosed above. Thirty-five thousand or more monoclonal antibodies against different human proteins are again arrayed on a chip and used to capture all cellular proteins in a first round of profiling. Then interactions of each of the bound cellular proteins can be simultaneously probed with any other appropriately labeled protein, nucleic acid or small molecule in a second round of affinity capture and visualization/quantitation. For instance, the secondary probe could be a signaling protein, a double stranded oligonucleotide from a promoter region, a small drug molecule, natural ligand or combinatorial chemistry product. Again quantitation of binding can be normalized for total amount of protein bound in any particular spot.

The above procedure is somewhat similar conceptually to arraying recombinant proteins. Recombinant proteins are easier to generate and array than monoclonal antibodies (6-7) and immobilized proteins will capture antibodies, for instance in sera, other proteins, nucleic acids and small molecules. However, recombinant proteins cannot be used for the purpose of expression profiling. In contrast, the multi-layered affinity interaction detection procedure disclosed herein offers distinct advantages over recombinant protein arrays for functional interaction analysis. The antibody chip/multi-layered affinity interaction detection procedure presents real cellular proteins for interaction profiling after a first round of cellular protein capture, whereas recombinant proteins may not give a complete and physiologically relevant picture of molecular interactions due to possible differences/problems in folding and absence of functionally relevant, e.g. inducible or disease-associated, post-translational modifications. Moreover, real cellular proteins captured by the multi-layered affinity interaction detection procedure may contain mutations that are highly relevant for drug screens or protein-protein interaction screens. A drug, protein or nucleic acid molecule may bind to a ‘wild type’ recombinant protein, but not to a mutant cellular one or vice versa.

A further refinement of the above technique is comparative analysis of protein pools from two different cell populations. In the same manner as described above for modification profiling, comparative functional interaction profiling could, for instance, yield information on which drugs differentially bind to which proteins from healthy versus diseased cells. Thus, a significant amount of information can be obtained efficiently from at least one protein-containing sample and can be used comparatively with information obtained in like manner from at least one other protein pool.

Generally, it is contemplated that an X number of detectably distinct affinity reagents, where X is an integer from 1 to N, can be used to detect an N number of post-translational modifications on a protein bound to a protein capture agent on a micro-array. Alternatively, if one of each of a second through N affinity reagents is applied separately to the labeled proteins captured in one each of the identical N arrays then the affinity reagents may be detectably identical. It is also contemplated that N microarrays may be used to detect an X number of post-translational modifications. The protein capture agents and the affinity reagents individually may be an anitibody or antibody fragment, recombinant proteins, nucleic acids, or phage particles. The affinity reagents may be labeled with a fluorophore, a radioisotope, may be detectable via chemiluminescence or may otherwise be detectable spectroscopically, such as using a fluorescently labeled secondary antibody or antibody fragment, or may be labeled with a non-fluorophore such as biotin, streptavidin, an enzyme, or nucleotide.

The following examples are given for the purpose of illustrating various embodiments of the invention and are not meant to limit the present invention in any fashion.

### EXAMPLE 1

Fabrication of Microarrays

Antibodies were printed on HydroGel slides (Perkin Elmer Life Sciences) using MicroSpot 2500 pins and a MicroGrid II arrayer (BioRobotocs). Printing ink was PBS containing 0.2% gelatin and 0.1% sodium azide. Printing concentration of each antibody was 200 μg/mL. Each antibody was spotted onto the array at least 5 times. The spacing between spots was 300 microns. Quality control of antibody deposition was performed using Cy⁵-labeled non-specific antibody. Quality control of retention of antibodies was performed using deposition of mouse IgG which was detected at the completion of experiments with Cy⁵-labeled goat anti-mouse antibody.

After completion of a printing cycle, arrays were incubated in the dark at room temperature and 65% relative humidity for at least 48 hrs. They were washed with PBST (PBS supplemented with 0.01 to 0.1% tween-20) for 30 min 3 times on an orbital shaker. Finally they were dipped in PBS, centrifuged at 1,000 rpm for 5 min, and left at 37° C. for a few minutes to allow them to dry completely. Arrays were stored in a non-condensing atmosphere at 4° C.

### EXAMPLE 2

Preparation of Protein Extract

Human leukemia cells, R10+ (glucophorin A positive), were grown in IMDM medium supplemented with 20% (v/v) heat inactivated fetal bovine serum and a penicillin-streptomycin mixture at 37° C. in 5% CO₂. Cells were collected and washed 4 times with ice-cold PBS without calcium and magnesium. The extraction buffer typically was carbonate buffer (pH 6.0 to 9.6) supplemented with EDTA (1 μM to 10 mM), IGEPAL (0.1 to 5%), NaF (1 μM to 10 mM), and Na₃VO₄ (1 μM to 10 mM). Ice-cold extraction buffer was added to cells. Proteins were extracted for 15 min on a rocking platform at 4° C. Cell debris was removed by centrifugation at 15,000 g for 30 min at 4° C. Protein content in the extract was determined using micro BCA reagent kit (Pierce).

### EXAMPLE 3

Labeling of Cellular Proteins with Fluorescent Tag

In a typical experiment, 2.6 mL of protein extract (protein concentration 0.1 to 0.5 mg/mL) was labeled with Cy⁵ fluorescent dye. NHS-ester activated Cy-dyes were from Amersham Biosciences. The dye (200 nmoles) was dissolved in a total volume of protein extract to be labeled and incubated in the dark at room temperature and gentle rocking for 30 minutes. Separation of non-incorporated dye was performed by gel-filtration on a Sephadex G-25 column (Amersham Biosciences) that was previously equilibrated with PBST. An equal volume of non-labeled protein extract was also applied to a G-25 column to exchange the buffer for incubation with arrays.

### EXAMPLE 4

Preparation of Microarrays for Incubation

Before an experiment, arrays were typically allowed to reach room temperature and blocked with PBST solution containing 10 mg/mL BSA for at least an hour with gentle agitation. Arrays were dipped in PBS, centrifuged at 1,000 rpm for 5 min and placed at 37° C. for a few minutes to allow them to dry.

### EXAMPLE 5

Detection of Protein Expression

Each microarray was incubated with 100 μL Cy⁵-labeled protein extract from R10 positive cells (0.2 mg/mL). Incubations were typically carried out using either microscope cover slips or 40×22 mm hybridization chambers (Grace Biolabs) for 1 h at 37° C. Protein extract was supplemented with 0.1% BSA. Upon completion of incubation, arrays were washed with PBST 4 times for 15 min at room temperature on an orbital shaker. They were dipped in PBS, centrifuged at 1,000 rpm for 5 min and left at 37° C. for a few minutes to allow them to dry. Arrays were scanned using a microarray scanner (Affymetrix).

### EXAMPLE 6

Detection of Proteins Phosphorylated at Tyr Residue

Each microarray was incubated with 100 uL non-labeled protein extract that has been passed through a G-25 column. Protein concentration typically was 0.2 mg/mL. Incubations were typically carried out using either microscope cover slips or 40×22 mm hybridization chambers (Grace Biolabs) for 1 h at 37° C. Protein extract was supplemented with 0.1% BSA. Upon completion of incubation, arrays were washed with PBST 4 times for 15 min at room temperature on an orbital shaker. Anti-phospho-Tyr antibody (PY100 from Cell Signaling Technology) was diluted 1:200 in antibody dilution buffer (PBST with 0.1% BSA) and 100 uL of this solution was incubated with each array for 1 h at 37° C. Arrays were washed with PBST 4 times for 15 min at room temperature on an orbital shaker. Cy⁵-labeled goat-anti-mouse antibody (Amersham Biosciences) was diluted 1:500 in the antibody dilution buffer and 100 uL of this solution was incubated with each array for 1 h at 37° C. Arrays were washed with PBST 4 times for 15 min at room temperature on an orbital shaker. They were dipped in PBS, centrifuged at 1,000 rpm for 5 min and left at 37° C. for a few minutes to allow them to dry. Arrays were scanned using a microarray scanner (Affymetrix).

### EXAMPLE 7

Data Analysis

The location of each protein on an array was determined by creating a Gal file with clone tracking option of BioRobotics software and importing it to GenePix Pro 4.0 software (Axon Instruments). Two separate images were imported into GenePix Pro and overlayed to be used in virtual MAID. Therefore, the first virtual layer represented total labeled protein bound to an array and second virtual layer represented Tyr phosphorylated protein bound to an array.

The fluorescence signal from each spot was determined as the average of the pixel intensities within the boundary outlined by software. Signal to local background (S/N, signal to noise) ratio was calculated for each spot. For comparison, S/N of MEK-1 protein that is known to be expressed in RT10+ cells but not phosphorylated at Tyr, was taken for 100%. Relative level of expression of a given protein was determined as a percentage of S/N of MEK-1 in the first virtual layer. Relative phosphorylation of a given protein at Tyr residue was determined as a percentage of S/N of MEK-1 in the second virtual layer.

### EXAMPLE 8

Phosphorylation at Tyr Residues of Raf-1 Protein in RT10+ Leukemia Cells

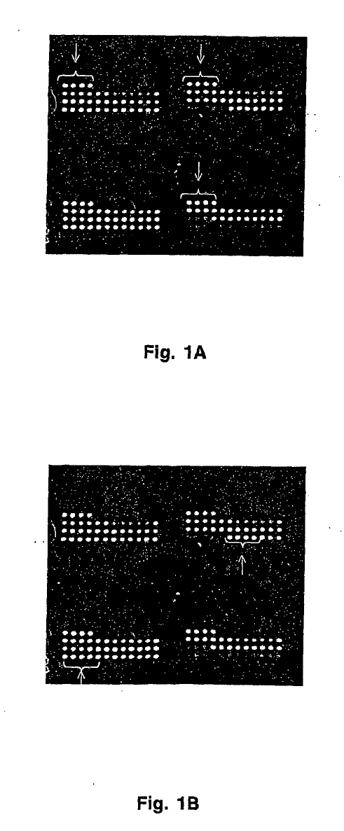

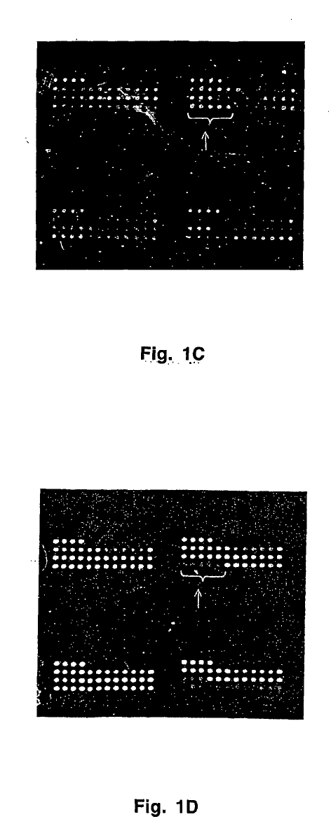

To determine post-translational phosphorylation at Tyr residues of proteins in RT10+ human leukemia cells, 21 antibodies were spotted on each array. Microarrays were constructed to contain 5 to 15 duplicate spots from each antibody. FIGS. 1A-1D depicts a typical array. Deposition of antibodies was confirmed by printing Cy⁵-labeled IgG (FIG. 1A). At the completion of an experiment, non-labeled mouse IgG on an array was visualized with Cy⁵-labeled goat anti-mouse IgG, confirming retention of capture antibodies on the surface of an array throughout the entire experiment (FIG. 1B).

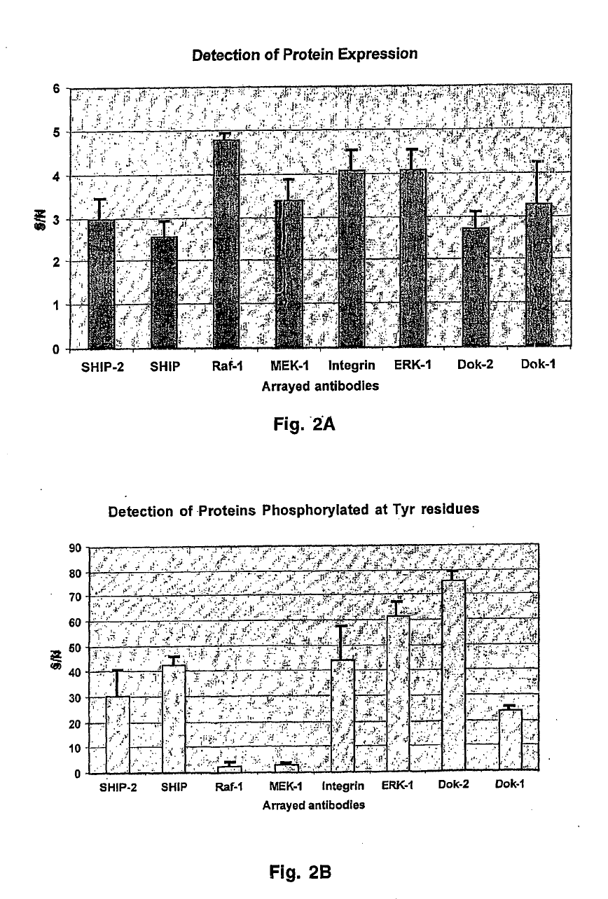

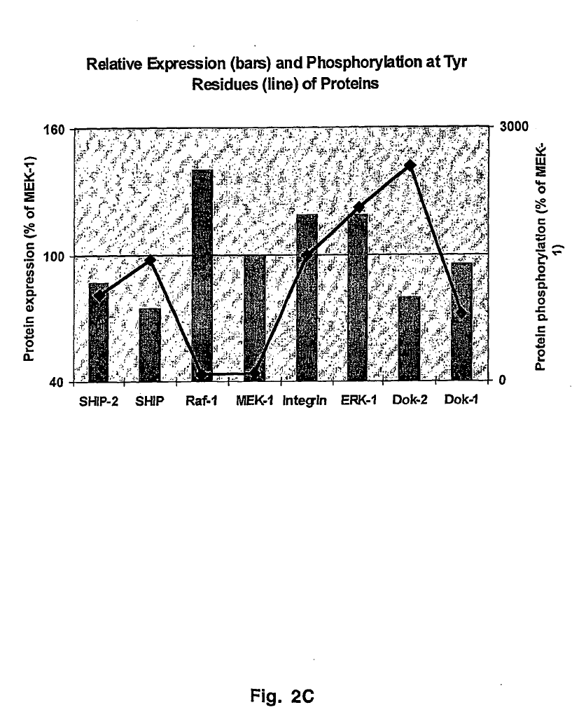

When Cy⁵-labeled protein extract was applied to the array, i.e., the first virtual layer, several proteins were visualized on the array (FIG. 1C and 2A). The highest signal was detected for spots corresponding to anti-Raf-1 antibody (shown by arrow in FIG. 1C). This was consistent with high level of expression of Raf-1 protein, a product of c-Raf oncogene, in RT10+ cells. Detection with anti-phosphotyrosine antibody, i.e., the second virtual layer, however revealed that Raf-1 was not phosphorylated at Tyr residues (FIG. 1C, shown by arrow and FIG. 2B). This was consistent with the knowledge that Raf-1 molecules are being phosphorylated at Ser, but not Tyr residues. The same situation was observed for MEK-1 protein that is also phosphorylated at Ser, but not Tyr.

### EXAMPLE 9

Phosphorylation at Tyr Residues of other Proteins in RT10+ Leukemia Cells

Several other proteins were determined to be both present and phosphorylated at tyrosines in the lysate from RT10+ cells (FIG. 1C,D and FIG. 2A, B). Because scanning of two virtual layers could be performed in 2 different channels or in the same channel but with 2 different PMT settings of a scanner, expression and phosphorylation of each protein was normalized against expression and phosphorylation of MEK-1. MEK-1 was selected as a “standard” because it was expressed but not phosphorylated at Tyr. FIG. 2C summarizes expression and phosphorylation patterns of proteins in RT10+ leukemia cells. The highest degree of Tyr phosphorylation was detected for Dok-2 protein, however, its relative expression was much lower than other Tyr phosphorylated proteins. Expression and phosphorylation patterns were consistent with those reported in literature.

The following references are cited herein:
1. Haab et al. (2001), Genome Biology 2(2): reviews 0004.1-0004.13.
2. Tomlinson and Holt (2001), Genome Biology 2(2): reviews 1004.1-1004.3.
3. Han et al. (2001), Nat. Biotechnol. 19:631-635.
4. Druker et al. (1994), J. Biol. Chem. 269:5387-5390.
5. Jaffrey et al. (2001), Nat. Cell Biol. 3:193-197.
6. Cahill, in Proteomics: A Trends Guide, July 2000, pp. 47-51.
7. MacBeath and Schreiber (2000), Science 289:1760-1763.

Any patents or publications mentioned in this specification are indicative of the levels of those skilled in the art to which the invention pertains. Further, these patents and publications are incorporated by reference herein to the same extent as if each individual publication was specifically and individually incorporated by reference.

One skilled in the art will appreciate readily that the present invention is well adapted to carry out the objects and obtain the ends and advantages mentioned, as well as those objects, ends and advantages inherent herein. The present examples, along with the methods, procedures, treatments, molecules, and specific compounds described herein are presently representative of preferred embodiments, are exemplary, and are not intended as limitations on the scope of the invention. Changes therein and other uses will occur to those skilled in the art which are encompassed within the spirit of the invention as defined by the scope of the claims.

1. A high throughput and quantitative method of analyzing post-translational protein modifications in a sample comprising the steps of:
a) preparing at least one of N identical arrays of immobilized protein capture agents, each of said capture agents binding specifically to a protein in said sample; and
b) performing in any order the steps comprising:
i) applying said proteins of the sample to at least one of said N arrays of immobilized protein capture agents; and
ii) binding said proteins of the sample to at least one of X detectable affinity reagents to label said proteins, wherein X is an integer from 1 to N and wherein each of said X detectable affinity reagents specifically recognizes one of N post-translational protein modifications; and
c) measuring a signal associated with said detectable affinity reagents, wherein quantitation of said signal from said X detectable affinity reagent(s) provides a high throughput and quantitative analysis of post-translational protein modifications in said sample.

2. The high throughput and quantitative method of claim 1, wherein step b comprises the order of:
binding said proteins of the sample to X detectable affinity reagents to label said proteins;
applying said labeled proteins to at least one of said N arrays of immobilized protein capture agents; and
binding said labeled proteins captured in at least one of said N arrays to (N-X) of said detectable affinity reagents.

3. The high throughput and quantitative method of claim 1, wherein step b comprises the order of:
binding said proteins of the sample to all of said X detectable affinity reagents to label said proteins; and
applying said labeled proteins to at least one of said N arrays of immobilized protein capture agents.

4. The high throughput and quantitative method of claim 1, wherein step b comprises the order of:
applying said proteins of the sample to at least one of said N arrays of immobilized protein capture agents; and
binding said captured proteins to all of X detectable affinity reagents.

5. The method of claim 1, wherein each of X affinity reagents are detectably distinct from said first affinity reagent and from each other.

6. The method of claim 1, wherein if one of each of a second through N affinity reagents is applied separately to said labeled proteins captured in one each of said identical N arrays, said affinity reagents are detectably identical.

7. The method of claim 1, wherein said protein capture agents are antibodies, antibody fragments, recombinant proteins, nucleic acids, or phage particles.

8. The method of claim 1, wherein said affinity reagents are antibodies, antibody fragments, recombinant proteins, nucleic acids, or phage particles.

9. The method of claim 8, wherein said affinity reagents are directly labeled with a detectable tag.

10. The method of claim 8, wherein said affinity reagents are labeled with a secondary detectable affinity reagent.

11. The method of claim 10, wherein said secondary affinity reagent is an antibody, an antibody fragment, a recombinant protein, a nucleic acid or page particle.

12. A high throughput and quantitative method of comparative analysis of post-translational protein modifications in different samples, comprising the steps of:
preparing an array of immobilized protein capture agents, each of said capture agents binding specifically to a protein in said samples;
incubating a first sample A with an affinity reagent, said affinity reagent labeled with a first detectable label, wherein said affinity reagent specifically recognizes a post-translational protein modification;
incubating a second sample B with said affinity reagent, said affinity reagent labeled with a second detectable label;
applying a mixture of said affinity reagent-labeled samples A and B to said array of immobilized protein capture agents;
quantifying relative signals from said first and said second detectable labels on said affinity reagents; and
calculating the ratios of said relative signals from said first and said second detectable labels, wherein said ratios correlate to the relative abundance of said post-translational modifications between said sample A and said sample B.

13. The method of claim 12, wherein said protein capture agents are antibodies, antibody fragments, recombinant proteins, nucleic acids or phage particles.

14. The method of claim 12, wherein said affinity reagent is an antibody, an antibody fragment, a recombinant protein, a nucleic acid, or a phage particle.

15. The method of claim 12, wherein said detectable labels are fluorophores, nucleic acids or enzymes.

16. A high throughput and quantitative method of comparative analysis of post-translational protein modifications in different samples, comprising the steps of:
preparing an array of immobilized protein capture agents, each of said capture agents binding specifically to a protein in said samples;
incubating a first sample A with an affinity reagent, said affinity reagent labeled with a first fluorophore, wherein said affinity reagent specifically recognizes a post-translational protein modification;
incubating a second sample B with said affinity reagent, said affinity reagent labeled with a second fluorophore;
applying a mixture of said affinity reagent-labeled samples A and B to said array of immobilized protein capture agents;
measuring the fluorescence emission of said first and said second fluorophores, and
calculating the ratios of relative fluorescence of said first and said second fluorophores, wherein said ratios correlate to the relative abundance of said post-translational modifications between said sample A and said sample B.

17. The method of claim 16, wherein said protein capture agents are antibodies, antibody fragments, recombinant proteins, nucleic acids or phage particles.

18. The method of claim 16, wherein said affinity reagent is an antibody, an antibody fragment, a recombinant protein, a nucleic acid, or a phage particle.

19. A high throughput and quantitative method of analyzing protein interactions, comprising the steps of:
preparing an array of immobilized protein capture agents, each of said capture agents binding specifically to a protein in said sample;
labeling the proteins in said sample with a first fluorophore;
applying the labeled proteins to said array of immobilized protein capture agents;
labeling molecules with a second fluorophore;
applying said labeled molecules to the labeled proteins captured on said array of immobilized capture agents, said molecules specifically binding to the labeled proteins captured on said array of immobilized capture agents; and
measuring the emission of said first and said second fluorophores, wherein the relative fluorescence of said first and of said second fluorophores correlates with an interaction of said molecules with the proteins thereby providing high throughput and quantitative analysis of the protein interactions.

20. The method of claim 19, wherein said protein capture agents are antibodies, antibody fragments, recombinant proteins, nucleic acids, or phage particles.

21. The method of claim 19, wherein said molecules are selected from the group consisting of protein molecules, small molecules, drug molecules and nucleic acid molecules.

22. A kit for a high throughput and quantitative method of analyzing post-translational protein modifications comprising:
at least one array of immobilized protein capture agents;
at least one buffer medium;
at least one affinity reagent, each of said affinity reagents recognizing a specific post-translational protein modification.

23. The kit of claim 22, wherein said protein capture agent(s) is an antibody, an antibody fragment, a recombinant protein, a nucleic acid or phage particles.

24. The kit of claim 22, wherein said affinity reagent(s) is an antibody, an antibody fragment, a recombinant protein, a nucleic acid or phage particles.

25. A kit for a high throughput and quantitative method of analyzing post-translational protein modifications comprising:
at least one array of immobilized protein capture agents; and
at least one buffer medium.

26. The kit of claim 25, wherein said protein capture agent(s) is an antibody, an antibody fragment, a recombinant protein, a nucleic acid or phage particles.

27. A kit for a high throughput and quantitative method of analyzing post-translational protein modifications comprising:
at least one affinity reagent, each of said affinity reagents recognizing a specific post-translational protein modification; and
at least one buffer medium.

28. The kit of claim 27, wherein said affinity reagent(s) is an antibody, an antibody fragment, a recombinant protein, a nucleic acid or phage particles.

29. A kit for a high throughput and quantitative method of analyzing post-translational protein modifications comprising:
at least one array of immobilized protein capture agents; and
at least one affinity reagent, each of said affinity reagents recognizing a specific post-translational protein modification.

30. The kit of claim 29, wherein said protein capture agent(s) is an antibody, an antibody fragment, a recombinant protein, a nucleic acid or phage particles.

31. The kit of claim 29, wherein said affinity reagent(s) is an antibody, an antibody fragment, a recombinant protein, a nucleic acid or phage particles.

32. A kit for a high throughput and quantitative method of analyzing post-translational protein modifications comprising:
a set of buffer media.
